(19) United States
(12) Patent Application Publication
(10) Pub. No.: US 2012/0071397 A1
(43) Pub. Date: Mar. 22, 2012

(54) Methods for Hematopoietic Stem Cell Transplantation

(75) Inventors: Kathleen E. Rodgers, Long Beach, CA (US); Gere S. diZerega, San Luis Obispo, CA (US)

(73) Assignee: University of Southern California, Los Angeles, CA (US)

(21) Appl. No.: 13/206,690

(22) Filed: Aug. 10, 2011

Related U.S. Application Data
(60) Provisional application No. 61/372,153, filed on Aug. 10, 2010.

(51) Int. Cl. A61K 38/08; A61P 31/14; C12N 5/0789; A61P 31/04

(52) U.S. Cl. 514/2.4; 514/3.7; 435/375

(57) ABSTRACT

The present invention provides methods for treating a subject undergoing hematopoietic stem cell transplantation, methods for increasing a subject's resistance to bacterial or viral infection during and/or following hematopoietic stem cell transplantation, and methods for treating norovirus infection.

### CROSS REFERENCE

This application claims priority to U.S. Provisional Patent Application Ser. No. 61/372,153, filed Aug. 10, 2010, incorporated by reference herein in its entirety.

### STATEMENT OF GOVERNMENT RIGHTS

This invention was made with U.S. government support under 5R01HL082722-02 awarded by the National Institute of Heart, Lung and Blood. The U.S. Government has certain rights in the invention.

### BACKGROUND OF THE INVENTION

Hematopoietic stem cell transplantation (HSCT) remains a risky procedure with many possible complications; it has traditionally been reserved for patients with life-threatening diseases. While occasionally used experimentally in nonmalignant and nonhematologic indications such as severe disabling auto-immune disease and cardiovascular disease, the risk of fatal complications appears too high to gain wider acceptance. Thus, improved methods of HCST are needed.

### SUMMARY OF THE INVENTION

In a first aspect, the present invention provides methods for treating a subject undergoing hematopoietic stem cell transplantation, comprising administering to

(a) a subject who has undergone or is undergoing myelosuppression therapy; and/or

(b) a donor who is donating hematopoietic stem cells to the subject,

a peptide comprising at least 5 contiguous amino acids of Asp-Arg-Val-Tyr-Ile-His-Pro (SEQ ID NO:1), or a pharmaceutical salt thereof. In various embodiments, the peptide comprises or consists of SEQ ID NO:1.

In a second aspect, the present invention provides methods for increasing a subject's resistance to bacterial or viral infection during and/or following hematopoietic stem cell transplantation, comprising administering to

(a) a subject who has undergone or is undergoing myelosuppression therapy; and/or

(b) a donor who is donating hematopoietic stem cells to the subject,

an amount effective of a peptide comprising at least 5 contiguous amino acids of Asp-Arg-Val-Tyr-Ile-His-Pro (SEQ ID NO:1), or a pharmaceutical salt thereof, to increase the subject's resistance to viral or bacterial infection during and/or following hematopoietic stem cell transplantation. In various embodiments, the peptide comprises or consists of SEQ ID NO:1.

In a third aspect, the present invention provides methods for treating a norovirus infection, comprising administering to a subject in need thereof an amount effective of a peptide comprising Asp-Arg-Val-Tyr-Ile-His-Pro (SEQ ID NO:1), or a pharmaceutical salt thereof, to treat the norovirus infection.

### DESCRIPTION OF THE FIGURES

FIG. 1. C57Bl/6 mice (n=40/group) were transplanted after myeloablation by total body irradiation (TBI) from Saline or A(1-7) treated donors and received saline or A(1-7) after transplant.

### DETAILED DESCRIPTION OF THE INVENTION

In a first aspect, the present invention provides methods for treating a subject undergoing hematopoietic stem cell transplantation, comprising administering to

(a) a subject who has completed myelosuppression therapy; and/or

(b) a donor who is donating hematopoietic stem cells to the subject;

a peptide comprising at least 5 contiguous amino acids of Asp-Arg-Val-Tyr-Ile-His-Pro (SEQ ID NO:1), or a pharmaceutical salt thereof.

In a second aspect, the present invention provides methods for increasing a subject's resistance to bacterial or viral infection during and/or following hematopoietic stem cell transplantation, comprising administering to

(a) a subject who has completed myelosuppression therapy; and/or

(b) a donor who is donating hematopoietic stem cells to the subject,

an amount effective of a peptide comprising at least 5 contiguous amino acids of Asp-Arg-Val-Tyr-Ile-His-Pro (SEQ ID NO:1), or a pharmaceutical salt thereof, to increase the subject's resistance to viral or bacterial infection during and/or following hematopoietic stem cell transplantation.

The inventors have surprisingly discovered that administration of A(1-7) to subjects undergoing hematopoietic stem cell transplantation (HSCT) after myelosuppression dramatically improved the subjects' resistance to viral or bacterial infection, and thus greatly improves HSCT safety. This is a significant finding, as subjects who have undergone myeloablation therapy are at a greatly increased risk of infection. This is because of the damage to the immune response during the conditioning regimen. Following myeloablation and prior to engraftment of the donor cells, the subject may go for several weeks without appreciable numbers of white blood cells, putting the subject at high risk of infection.

The subject is any suitable mammalian subject who has completed myelosuppression therapy, preferably a human subject.

As is known in the art, “A(1-7)” is a peptide having the amino acid sequence Asp-Arg-Val-Tyr-Ile-His-Pro (SEQ ID NO:1). In various embodiments, the peptide comprises or consists of Asp-Arg-Val-Tyr-Ile (A(1-5) (SEQ ID NO:2)), Asp-Arg-Val-Tyr-Ile-His (A(1-6) (SEQ ID NO:3)), or A(1-7).

Transplant may occur in any suitable subject, including but not limited to those with neoplastic disorders (particularly hematological malignancies) and non-neoplastic disorders including aplastic anemia, autoimmune disease, immunodeficiency and inborn errors of metabolism, such as severe combined immunodeficiency, chronic granulomatous disease and X linked hypohirotic ectodermal dysplasia and immunodeficiency.

As used herein, “Hematopoietic stem cell transplantation” is the transplantation of blood stem cells derived from the bone marrow (in this case known as bone marrow transplantation), blood (such as peripheral blood and umbilical cord blood), or amniotic fluid. Collecting peripheral blood stem cells provides a bigger graft, does not require that the donor be subjected to general anesthesia to collect the graft, results in a shorter time to engraftment, and may provide for a lower long-term relapse rate. The invention is appropriate for use in individuals that are undergoing transplant with hematopoietic progenitors of autologous, allogeneic or neonatal origin.

Autologous HSCT comprises the extraction of HSC from the subject and freezing of the harvested HSC. After myeloablation, the subject's stored HSC are transplanted into the subject. Thus, in this embodiment there is no donor involved. Allogeneic HSCT involves HSC obtained from an allogeneic HSC donor who has an HLA type that matches the subject.

As used herein, “myelosuppression therapy” is a preparative therapy designed to eradicate some or all of the subject's hematopoietic cells prior to transplant. In one embodiment, “myeloablation” is the severe or complete depletion of HSC by the administration of high doses of chemotherapy and/or radiation therapy prior to HCST. This treatment severely impacts the myeloproliferative function of the hematopoietic system. Any suitable myeloablation techniques may be used. For example, for allogeneic transplants a combination of cyclophosphamide with busulfan or TBI is commonly employed. Autologous transplants may also use similar conditioning regimens, but many other chemotherapy combinations can be used depending on the disease. In another embodiment, “non-myeloablative” therapy is used, in which lower doses or chemotherapy and/or radiation therapy are used that do not eradicate all of the hematopoietic cells.

The subject is one that has completed myeloablation, Myeloablation can occur by any treatment effective at reducing the myeloproliferative activity of the hematopoetic system including but not limited to chemotherapy, radiation therapy, or both. In one preferred embodiment, the administering to the subject occurs within 0, 1, 2, 3, 4, 5, 6, or 7 days of completing myeloablation. In another embodiment, that can be combined with any of the above embodiments, the administering begins place simultaneously with HSCT, and may include a single administration, or multiple administrations (2, 3, 4, 5, 6, 7, 8, 9, 10, etc.) as deemed appropriate.

In preferred embodiments, the peptide is administered (to subject or donor) in a dosage of 10 μg/kg/day, 50 ug/day μg/kg/day, 100 μg/kg/day, 250 μg/kg/day, 500 μg/kg/day, 1000 μg/kg/day or more. In various embodiments, the amount of A(1-7) or pharmaceutical salt thereof is sufficient to provide a dosage to a patient of between 0.01 μg/kg and 10 mg/kg; 0.1 μg/kg and 5 mg/kg; 0.1 μg/kg and 1000 μg/kg; 0.1 μg/kg and 900 μg/kg; 0.1 μg/kg and 900 μg/kg; 0.1 μg/kg and 800 μg/kg; 0.1 μg/kg and 700 μg/kg; 0.1 μg/kg and 600 μg/kg; 0.1 μg/kg and 500 μg/kg; or 0.1 μg/kg and 400 μg/kg. Peptide can be administered as often as appropriate to achieve the desired result, including but not limited once per day, twice per day, every other day, three times per week, twice per week, or once per week. In a preferred embodiment, peptide is administered until the subject is considered immunocompetent (e.g., able to produce a medically useful or meaningful immune response following exposure to an antigen).

In one embodiment, only the subject is treated with the peptide. In a preferred embodiment, the method further comprises contacting donor HSC to be transplanted with the peptide prior to transplantation into the subject, wherein such contacting may occur in vivo (ie: administering to an allogenic transplant donor) or in vitro (ie: culturing/expanding donor cells in vitro in the presence of peptide in any type of transplant). When the contacting occurs in vitro, any suitable concentration of peptide can be used. In a preferred embodiment, peptide is used in vitro at between 0.001 mg/ml to 10 mg/ml, more preferably between 0.005 mg/ml and 7.5 mg/ml, or between 0.01 mg/ml and 3 mg/ml.

In a third aspect, the present invention provides methods for treating a norovirus infection, comprising administering to a subject in need thereof an amount effective of a peptide comprising Asp-Arg-Val-Tyr-Ile-His-Pro (SEQ ID NO:1), or a pharmaceutical salt thereof, to treat the norovirus infection. Norovirus is an RNA virus that is extremely infectious and causes approximately 90% of epidemic non-bacterial outbreaks of gastroenteritis around the world and may be responsible for 50% of all food-borne outbreaks of gastroenteritis in the US. Norovirus affects people of all ages. The viruses are transmitted by fecally contaminated food or water, by person-to-person contact, and via aerosolization of the virus and subsequent contamination of surfaces. Outbreaks of norovirus infection often occur in closed or semi-closed communities, such as long-term care facilities, overnight camps, hospitals, prisons, dormitories, and cruise ships where the infection spreads very rapidly.

The subject is any suitable mammalian subject infected with norovirus, preferably a human subject.

In one embodiment, the methods comprise treating subjects infected with Noroviruses belonging to genogroups genogroup I (GI) includes Norwalk virus, Desert Shield virus and Southampton virus and II (GII), which includes Bristol virus, Lordsdale virus, Toronto virus, Mexico virus, Hawaii virus and Snow Mountain virus. These genogroups are commonly isolated in cases of acute gastroenteritis and are most common in humans. In another embodiment, the methods comprise treating a norovirus infection comprising one or more serotypes, strains, and isolates selected from the group consisting of:

Norwalk virus [M87661] (Hu/NLV/NV/1968/US)

Hawaii virus [U07611] (Hu/NLV/HV/1971/US)

Snow Mountain virus [L23831] (Hu/NLV/SMV/1976/US)

Mexico virus [U22498] (Hu/NLV/MX/1989/MX)

Desert Shield virus [U04469] (Hu/NLV/DSV395/1990/SR)

Southampton virus [L07418] (Hu/NLV/SHV/1991/UK); and

Lordsdale virus [X86557] (Hu/NLV/LD/1993/UK).

Symptoms of norovirus infection include, but are not limited to, gastroenteritis, nausea, forceful vomiting, watery diarrhea, and abdominal pain. Thus, the methods comprise treating a subject presenting with such symptoms and who are at risk of norovirus infection, such as those subjects in closed or semi-closed communities, such as long-term care facilities, overnight camps, hospitals, prisons, dormitories, and cruise ships, or who otherwise are at risk for norovirus infection. In another embodiment, the methods comprise treating a subject that has been diagnosed as having been infected with norovirus, such as by a polymerase chain reaction (PCR) assay.

As used herein, “treat” or “treating” norovirus infection means accomplishing one or more of the following: (a) reducing norovirus viral titer in the subject; (b) limiting any increase of norovirus viral titer in the subject; (c) reducing the severity of norovirus symptoms; (d) limiting or preventing development of norovirus symptoms after infection; (e) inhibiting worsening of norovirus symptoms; and (f) limiting or preventing recurrence of norovirus symptoms in subjects that were previously symptomatic for influenza infection.

In preferred embodiments of this third aspect, the peptide is administered in a dosage of 10 μg/kg/day, 50 ug/day μg/kg/day, 100 μg/kg/day, 250 μg/kg/day, 500 μg/kg/day, 1000 μg/kg/day or more. In various embodiments, the amount of A(1-7) or pharmaceutical salt thereof is sufficient to provide a dosage to a patient of between 0.01 μg/kg and 10 mg/kg; 0.1 μg/kg and 5 mg/kg; 0.1 μg/kg and 1000 μg/kg; 0.1 μg/kg and 900 μg/kg; 0.1 μg/kg and 900 μg/kg; 0.1 μg/kg and 800 μg/kg; 0.1 μg/kg and 700 μg/kg; 0.1 μg/kg and 600 μg/kg; 0.1 μg/kg and 500 μg/kg; or 0.1 μg/kg and 400 μg/kg. Peptide can be administered as often as appropriate to achieve the desired result, including but not limited once per day, twice per day, every other day, three times per week, twice per week, or once per week.

In all aspects and embodiments of the invention, suitable acids which are capable of forming salts with A(1-7) include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming salts with A(1-7) include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

Pharmaceutical compositions for use in the methods of the invention may be made up in a solid form (including granules, powders or suppositories) or in a liquid form (e.g., solutions, suspensions, or emulsions). The pharmaceutical compositions may be applied in a variety of solutions. Suitable solutions for use in accordance with the invention are sterile, dissolve sufficient amounts of the A(1-7), and are not harmful for the proposed application. In this regard, the compounds of the present invention are very stable but are hydrolyzed by strong acids and bases. The compounds of the present invention are soluble in organic solvents and in aqueous solutions at pH 5-8. The pharmaceutical compositions may be subjected to conventional pharmaceutical operations such as sterilization and/or may contain conventional adjuvants.

In another embodiment of all aspects and embodiments of the invention, the A(1-7) or salt thereof is prepared as a stable lyophilized peptide formulation that can be reconstituted with a suitable diluent to generate a reconstituted pharmaceutical compositions of the invention that are suitable for subcutaneous administration When reconstituted with a diluent comprising a preservative (such as bacteriostatic water for injection), the reconstituted formulation may be used as a multi-use formulation. Such a formulation is useful, for example, where the subject requires frequent subcutaneous administrations of A(1-7). The advantage of a multi-use formulation is that it facilitates ease of use for the patient, reduces waste by allowing complete use of vial contents, and results in a significant cost savings for the manufacturer since several doses are packaged in a single vial (lower filling and shipping costs). Such reconstituted formulations would also be suitable for use with other types of parenteral administration.

For administration, the pharmaceutical compositions are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, hydroxyethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art. Methods for the production of these formulations with the peptides or pharmaceutical compositions of the present invention are apparent to those of ordinary skill in the art.

In other embodiments of all aspects of the invention, the pharmaceutical compositions of the present invention may further comprise one or more other therapeutics as needed by a given subject.

A(1-7) or salts thereof can further be derivatized to provide enhanced half-life, for example, by linking to polyethylene glycol. The A(1-7) or salts thereof may comprise L-amino acids, D-amino acids (which are resistant to L-amino acid-specific proteases in vivo), a combination of D- and L-amino acids, and various “designer” amino acids (e.g., β-methyl amino acids, Cα-methyl amino acids, and Nα-methyl amino acids, etc.) to convey special properties. Synthetic amino acids include norleucine for isoleucine. In addition, the A(1-7) or salts thereof can have peptidomimetic bonds. For example, an A(1-7) peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such polypeptides are resistant to protease activity, and possess an extended half-live in vivo.

A(1-7) or salts thereof may be chemically synthesized or recombinantly expressed, each of which can be accomplished using standard methods in the art.

The A(1-7) or salts thereof can be administered by any suitable route, including but not limited to dermal, subcutaneous, intradermal, transdermal (for example, by slow-release polymers), intramuscular, intraperitoneal, intravenous, oral, aural, epidural, anal or vaginal (for example, by suppositories), and intranasal routes, infusion or bolus injection, or absorption through epithelial or mucocutaneous linings.

### Example

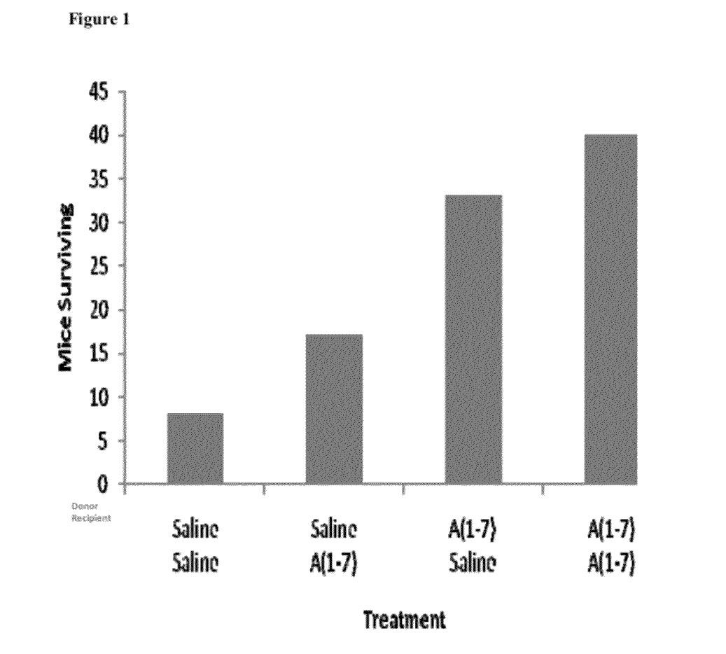

The present study demonstrates unexpected evidence of immune stimulation upon treatment with A(1-7). C56Bl/6 mice underwent 1100 TBI in split doses separated by 3 hours. Two hours after TBI, the mice received 2×106 syngeneic bone marrow cells by intravenous injection. For treatment, either donors (14 days prior to donation), recipients (starting on the day of transplant) or both received 500 μg/kg/day A(1-7) by subcutaneous injection During bone marrow transplant (BMT), recipients are immune suppressed due to myeloablation. Mice are normally kept in pathogen-free environments during BMT to reduce mortality due to infection. In this study involving BMT, mice began to die from unexplained causes on Day 3 after transplant (stopping on day 12). Treatment with antibiotics (240 mg/1 sulfamethoxazole, 48 mg/1 trimethoprim) did not stop the deaths. Surprisingly, it was found that treatment of either donor, recipient or both with A(1-7) resulted in improved survival, with all surviving if both were treated (FIG. 1). A viral infection was suspected as antibiotic treatment did not prevent mortality, and the time course of deaths. Evaluation of the serum from surviving animals, as well as rack-matched sentinels, showed antibodies indicating exposure to virulent norovirus supporting the hypothesis that mortality was due to viral infection. These data provides evidence that A(1-7) increases host resistance to pathogens.

We claim:

1. A method for treating a subject undergoing hematopoietic stem cell transplantation, comprising administering to
(a) a subject who has completed myelosuppression therapy; and/or
(b) a donor who is donating hematopoietic stem cells to the subject,
a peptide comprising Asp-Arg-Val-Tyr-Ile-His-Pro (SEQ ID NO:1), or a pharmaceutical salt thereof.

2. The method of claim 1, wherein the peptide consists of SEQ ID NO:1.

3. The method of claim 1, wherein the subject is suffering from a neoplastic disorder, aplastic anemia, autoimmune disease, immunodeficiency, or an inborn error of metabolism.

4. The method of claim 1, wherein the hematopoietic stem cell transplantation comprises transplantation of blood stem cells derived from bone marrow.

5. The method of claim 1, wherein the hematopoietic stem cell transplantation comprises transplantation of blood stem cells derived from blood.

6. The method of claim 1, wherein the hematopoietic stem cell transplantation comprises transplantation of blood stem cells derived from amniotic fluid.

7. The method of claim 1, wherein the myelosuppression therapy comprise severe or complete depletion of hematopoietic stem cells by administering high doses of chemotherapy and/or radiation therapy prior to hematopoietic stem cell transplantation.

8. The method of claim 1, wherein the peptide is administered to the subject.

9. The method of claim 1, wherein the peptide is administered to the donor.

10. The method of claim 1, wherein the peptide is administered to the subject and the donor.

11. The method of claim 1, wherein contacting donor hematopoietic stem cells with the peptide occurs in vivo.

12. The method of claim 1, wherein contacting donor hematopoietic stem cells with the peptide occurs in vitro.

13. A method for increasing a subject's resistance to bacterial or viral infection during and/or following hematopoietic stem cell transplantation, comprising administering to
(a) a subject who has completed myelosuppression therapy; and/or
(b) a donor who is donating hematopoietic stem cells to the subject,
an amount effective of a peptide comprising Asp-Arg-Val-Tyr-Ile-His-Pro (SEQ ID NO:1), or a pharmaceutical salt thereof, to increase the subject's resistance to viral or bacterial infection during and/or following hematopoietic stem cell transplantation.

14. The method of claim 13, wherein the peptide consists of SEQ ID NO:1.

15. The method of claim 13, wherein the subject is suffering from a neoplastic disorder, aplastic anemia, autoimmune disease, immunodeficiency, or an inborn error of metabolism.

16. The method of claim 13, wherein the hematopoietic stem cell transplantation comprises transplantation of blood stem cells derived from bone marrow.

17. The method of claim 13, wherein the hematopoietic stem cell transplantation comprises transplantation of blood stem cells derived from blood.

18. The method of claim 13, wherein the hematopoietic stem cell transplantation comprises transplantation of blood stem cells derived from amniotic fluid.

19. The method of claim 13, wherein the myelosuppression therapy comprise severe or complete depletion of hematopoietic stem cells by administering high doses of chemotherapy and/or radiation therapy prior to hematopoietic stem cell transplantation.

20. The method of claim 13, wherein the peptide is administered to the subject.

21. The method of claim 13, wherein the peptide is administered to the donor.

22. The method of claim 13, wherein the peptide is administered to the subject and the donor.

23. The method of claim 13, wherein contacting donor hematopoietic stem cells with the peptide occurs in vivo.

24. The method of claim 13, wherein contacting donor hematopoietic stem cells with the peptide occurs in vitro.

25. A method for treating a norovirus infection, comprising administering to a subject in need thereof an amount effective of a peptide comprising Asp-Arg-Val-Tyr-Ile-His-Pro (SEQ ID NO:1), or a pharmaceutical salt thereof, to treat the norovirus infection.


SEQUENCE LISTING (3 sequences)

SEQ ID NO: 1 - protein, 7 aa, Artificial Sequence
  Synthetic
DRVYIHP

SEQ ID NO: 2 - protein, 5 aa, Artificial Sequence
  Synthetic
DRVYI

SEQ ID NO: 3 - protein, 6 aa, Artificial Sequence
  Synthetic
DRVYIH